(19) United States
(12) Patent Application Publication
(10) Pub. No.: US 2010/0087493 A1
(43) Pub. Date: Apr. 8, 2010

(54) METHOD FOR IMPROVING THE PHARMACOKINETICS OF DRUGS METABOLIZED BY UGT2B10

(76) Inventors: Sanna Kaivosaari, Nummela (FI); Moshe Finel, Helsinki (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/524,771

(22) PCT Filed: Jan. 29, 2008
(86) PCT No.: PCT/FI2008/000017

§ 371 Date: Sep. 1, 2009

Related U.S. Application Data
(60) Provisional application No. 60/897,825, filed on Jan. 29, 2007.

(51) Int. Cl. A61K 31/465; C12Q 1/48; C12Q 1/02

(52) U.S. Cl. 514/343; 435/15; 435/29

(57) ABSTRACT

A method for modifying the pharmacokinetics of a pharmacologically active agent that undergoes direct N-glucuronidation by UDP-glucuronosyltransferase isoenzyme UGT2B10 in a human subject comprising administering an effective amount of an UGT2B10 modulator to said human subject. A method for identifying compounds which are directly metabolized by UGT2B10 or which act as UGT2B10 modulators is also disclosed.

### TECHNICAL FIELD

The present invention relates to optimization of drug pharmacokinetics. In particular, the present invention relates to a method for modifying the pharmacokinetics of a drug that is metabolized by direct N-glucuronidation via the human UDP-glucuronosyltransferase isoenzyme UGT2B10. The present invention also relates to a method for identifying compounds that are directly glucuronidated by UGT2B10 and to method for identifying compounds which act as UGT2B10 modulators.

### BACKGROUND OF THE INVENTION

Hepatic metabolism is the principal elimination mechanism for the majority of drugs in humans. One such metabolic pathway is glucuronidation catalyzed by a family of membrane-bound UDP-glucuronosyltransferase enzymes (UGTs). Numerous functional UGT genes have been characterised in the human genome and they are divided into two main subfamilies, UGT1A and UGT2B. The nomenclature of UGT superfamily is reviewed in Mackenzie P. I. et al., Pharmacogenetics and Genomics, 15 (10), 677-685, 2005. Many members of the UGT superfamily are expressed in the human liver as well as in several extrahepatic tissues such as the gastrointestinal tract, kidney and lungs (Tukey R. H. and Strassburg C. P., Annual Review of Pharmacology and Toxicology, 40, 581-616, 2000).

In glucuronidation, the glucuronic acid moiety of the endogenous cofactor UDP-glucuronic acid is transferred to the aglycone substrate, usually a small lipophilic compound. The resulting glucuronide is generally more water-soluble, less toxic and more easily excreted from the body than the parent compound. The compounds that undergo glucuronidation include endogenous substrates, such as steroids, bile acids, bilirubin, hormones, dietary constituents, and thousands of xenobiotics that include drugs, environmental toxicants, and carcinogens. A common feature of UGT substrates is the presence of a nucleophilic functional group, mainly nucleophilic functional group of oxygen or nitrogen, in their chemical structure. However, other nucleophilic functional groups, e.g. those involving sulphur or carbon, have also been shown to undergo glucuronidation. Although glucuronidation often occurs after cytochrome P450 (CYP) dependent oxidative metabolism, many compounds, including drugs, do not require prior oxidation, because they already possess suitable functional groups that can be glucuronidated. Thus, UGTs play a key role in elimination of compounds that undergo direct glucuronidation, such as non-steroidal anti-inflammatories, opioids, antihistamines, antipsychotics and antidepressive drugs.

Drugs that are extensively metabolized by UGTs may suffer from unfavourable pharmacokinetics and bioavailability, leading to more frequent or higher drug doses than would otherwise be necessary or desirable. Administration of such drugs together with an agent that would inhibit UGT metabolism can potentially improve the pharmacokinetics of the drug and permit a significant reduction in the frequency and/or amount of the drug doses. Alternatively, an agent that would accelerate UGT metabolism would be desirable for improved elimination of environmental toxicants and carcinogens in subjects who are in risk of such exposure. However, modifying the UGT metabolism of a given drug is complicated by the genetic multiplicity of the UGT family and the current poor understanding of the exact contribution of individual UGTs to the metabolism of a given drug.

N-glucuronidation refers to conjugation of glucuronic acid to a nucleophilic nitrogen atom of the aglycone substrate. A nucleophilic nitrogen atom can be present in various chemical structures. For example, aryl- and alkylamines, sulfonamides and various aromatic or aliphatic heterocyclic compounds having one or more nitrogen atoms as heteroatom, were reported to undergo N-glucuronidation in a number of animal species and, particularly, in humans. Distinct species differences have been observed in N-glucuronidation among certain substrates, for example, the preferential glucuronidation of tertiary amines to form quaternary glucuronides by humans and higher primates (chimpanzees) (Chiu S. H. and Huskey S. W., Drug Metab Dispos. 26(9), 838-47, 1998).

Nicotine and its primary oxidation metabolite, cotinine, are examples of compounds that undergo direct N-glucuronidation at the aromatic nitrogen. It has been reported that the reaction is catalyzed by one or more of the human liver UGTs. Nevertheless, previous attempts to identify the individual UGTs prominent in N-glucuronidation of nicotine and cotinine have been inconclusive (Ghoseh O. and Hawes E. M., Drug Metabolism and Disposition, 30, 991-996, 2002; Nakajima M. et al., Drug Metabolism and Disposition, 30, 1484-1490, 2002). Many questions remained unanswered, even though it has been suggested that UGT1A4 would be responsible for hepatic glucuronidation of nicotine and cotinine (Nakajima M. and Yokoi T., Drug Metab. Pharmacokinet., 20 (4), 227-235, 2005; Kuehl G. E. and Murphy S. E., Drug Metabolism and Disposition, 31, 1361-1368, 2003).

Better understanding of the roles of UGT family members in drug metabolism would assist in developing drug therapies with improved pharmacokinetics. Nicotine, for example, has been suggested as potential medication in the treatment of Parkinson's disease, Alzheimer's disease and ulcerative colitis. Reducing the elimination rate of nicotine would therefore be useful as a therapeutic approach to smoking reduction and cessation as well as in the treatment of Parkinson's disease, Alzheimer's disease and ulcerative colitis.

### SUMMARY OF THE INVENTION

We have unexpectedly found that a little studied human UGT isoenzyme, namely UGT2B10, rather than UGT1A4, is mainly responsible for the hepatic N-glucuronidation of nicotine and cotinine. This indicates that UGT2B10 potentially plays an important role in the glucuronidation of various pharmacologically active agents that undergo direct N-glucuronidation. It was also found that the activity of recombinant UGT2B10 often declines sharply during the isolation of microsomal membranes from the cells in which the enzyme was expressed. Skipping the step of microsomal membrane isolation and switching to the use of cell homogenates revealed that the activity of recombinant UGT2B10 is much higher than previously assumed, particularly in catalyzing N-glucuronidation. In addition, it was found that N-glucuronidation of nicotine can be inhibited by agents that act as UGT2B10 modulators. Thus, improvements in pharmacokinetic parameters such as peak plasma concentration (Cₘₐₓ), minimum plasma concentration (Cₘᵢₙ), area under curve of plasma concentration versus time (AUC) and/or half-life (T_{1/2}) can be achieved by administering a drug that undergoes direct N-glucuronidation via UGT2B10 together with an agent that acts as an UGT2B10 modulator. Such co-administration can be expected to provide enhanced drug efficacy, as the biotransformation of the drug is inhibited and the total amount of drug systemically available over time is increased.

### DETAILED DESCRIPTION OF THE INVENTION

The present invention provides, as one aspect of the invention, a method for modifying the pharmacokinetics of a pharmacologically active agent that undergoes direct N-glucuronidation by the UDP-glucuronosyltransferase UGT2B10 in a human subject, which method comprises administering an effective amount of an UGT2B10 modulator to said human subject. According to one preferred embodiment of the invention, said pharmacologically active agent undergoes direct N-glucuronidation predominantly by UGT2B10.

The present invention also provides, as another aspect of the invention, a pharmaceutical combination product comprising a pharmacologically active agent that is useful for the treatment or prevention of a disease or a condition and that undergoes direct N-glucuronidation by UDP-glucuronosyltransferase UGT2B10, together with an UGT2B10 modulator in an amount that provides improved pharmacokinetics of said agent. According to one preferred embodiment of the invention, said pharmacologically active agent undergoes direct N-glucuronidation predominantly by UGT2B10.

The present invention also provides, as another aspect of the invention, a method for identifying a compound which is directly metabolized by UGT2B10 which method comprises

(a) providing a homogenate of recombinant cells that express UGT2B10,
(b) incubating the compound to be tested with said homogenate together with a glucuronic acid source, and
(c) determining whether N-glucuronidated derivative of the compound was formed.

The present invention also provides, as yet another aspect of the invention, a method for identifying a compound which acts as UGT2B10 modulator which method comprises

(a) providing a homogenate of recombinant cells that express UGT2B10,
(b) incubating a UGT2B10 substrate with said homogenate together with a glucuronic acid source in the presence and in the absence of the compound to be tested,
(c) quantifying the amount of N-glucuronidated derivative of said UGT2B10 substrate that was formed in the presence and in the absence of the tested compound, and
(d) determining whether the N-glucuronidation of said UGT2B10 substrate was inhibited or activated by the tested compound.

The term “UGT2B10 modulator” means here a compound which can modify, e.g. inhibit or enhance, the UGT2B10 activity towards a pharmacologically active agent. An UGT2B10 modulator that inhibits the UGT2B10 activity towards a pharmacologically active agent is referred here as a UGT2B10 inhibitor. Similarly, an UGT2B10 modulator that enhances the UGT2B10 activity towards a pharmacologically active agent is referred here as a UGT2B10 activator. Thus, a UGT2B10 inhibitor is expected to increase the total amount of a pharmacologically active agent systemically available over time, whereas a UGT2B10 activator is expected to decrease it. An UGT2B10 inhibitor may inhibit UGT2B10 activity by various mechanisms including competitive, non-competitive, uncompetitive, mixed or irreversible inhibition.

The term “effective amount of an UGT2B10 modulator” means here an amount of UGT2B10 modulator sufficient to provide improved pharmacokinetics of the pharmacologically active agent in the presence of UGT2B10 modulator compared to the pharmacokinetics of the pharmacologically active agent in the absence of the UGT2B10 modulator. In case the pharmacologically active agent is a drug (i.e. is useful for the treatment or prevention of a disease or a condition) the improved pharmacokinetics generally relates to an increase in the total amount of drug systemically available over time. Such improved pharmacokinetics may comprise, for example, increased peak plasma concentration (Cₘₐₓ) and/or minimum plasma concentration (Cₘᵢₙ), increased area under curve of plasma concentration versus time (AUC) and/or increased half-life (T_{1/2}), or reduced inter-individual or intra-individual variability in these values. For example, the UGT2B10 modulator may provide Cₘₐₓ, Cₘᵢₙ, AUC and/or T_{1/2} values for the active agent that are at least 1.1 fold, at least 1.2 fold, at least 1.3 fold or at least 1.5 fold, compared to values obtained in the absence of UGT2B10 modulator.

The pharmacologically active agent that undergoes direct N-glucuronidation by UGT2B10 is preferably a compound having a nucleophilic nitrogen atom in its chemical structure. These include primary, secondary and tertiary aryl- and alkylamines, sulfonamides and aromatic or aliphatic heterocyclic compounds having one or more nitrogen atoms as heteroatom.

For the purpose of identifying compounds which are directly metabolized by UGT2B10, recombinant cells that express UGT2B10 are prepared according to known methods. For example, recombinant UGT2B10 can be expressed in baculovirus-infected insect cells as described e.g. in Kurkela, M. et al., Journal of Biological Chemistry, 278(6), 3536-3544, 2003. The cells expressing UGT2B10 are suspended in water and homogenized using known procedures. Incubation mixture is prepared by mixing recombinant UGT2B10 homogenate and uridine-5-diphospho-glucuronic acid (UDP-GA) as a glucuronic acid source in a suitable buffer such as phosphate buffer pH 7.4. The compound to be tested for its potential to be metabolized by UGT2B10 is added to the mixture, which is incubated at 37° C. for a period needed for the glucuronidation reaction to occur, typically from 0.5 to 2 hours. The reaction is terminated by protein precipitation, e.g. by addition of trifluoroacetic acid, or acetonitrile if the formed N-glucuronide is expected to be acid-labile, and cooling. Following centrifugation, the supernatants are analysed for the presence of glucuronidated derivative of the test compound, wherein any suitable analyzing method can be used. High pressure liquid chromatography followed by mass spectrometry is the preferred method. The glucuronides are quantified using authentic glucuronide standards or another suitable methods such as substrate depletion. The enzyme activity of UGT2B10 towards the tested compound is calculated as the amount of glucuronide formed (pmol) divided by incubation time (min) and protein amount (mg/incubation). Example 1 of the present application describes a procedure suitable for identifying compounds that are glucuronidated by UGT2B10.

For the purpose of testing the selectivity of UGT2B10 over other UGTs for the tested compound, recombinant cells that express UGT enzymes are prepared as described above. Human liver microsomes (HLM) are obtained e.g. from BD Biosciences, Woburn, Mass., USA. The recombinant UGT enzymes are used after the isolation of the microsomal membranes by centrifugation, which is a common preparation step for UGT enzymes. However, UGT2B10 is used as cell homogenates as the activity of this enzyme sharply declines upon the isolation of the microsomal membranes. Incubation mixture is prepared by mixing the recombinant UGT enzyme or HLM and uridine-5-diphosphoglucuronic acid (UDP-GA) as a glucuronic acid source in a suitable buffer such as phosphate buffer pH 7.4. The compound to be tested for its potential glucuronidation by each UGT or HLM is added to the mixture at various concentrations (e.g. 0.002-2 mM). The mixture is incubated at 37° C. for a period needed for the glucuronidation reaction to occur, typically from 0.5 to 2 hours. The reaction is terminated as described above. Following centrifugation, the supernatants are analyzed and quantified for the presence of glucuronidated derivative of the test compound as described above. The enzyme activities for UGTs and human liver microsomes at various concentrations of the tested compound are calculated as the amount of glucuronide formed (mol) divided by incubation time (min) and protein amount (mg/incubation). Enzyme kinetic parameter Kₘ is estimated by fitting the enzyme activity data to Michaelis-Menten equation using suitable software such as SigmaPlot Enzyme Kinetics Module v. 1.1 (SPSS Inc., Chicago, Ill.). The Kₘ value is used as a measure for selectivity. According to one preferred embodiment of the invention, the compound identified as a UGT2B10 substrate undergoes direct N-glucuronidation predominantly by UGT2B10, wherein the term “predominantly” indicates that the Kₘ value of the tested compound calculated for UGT2B10 is less than the calculated Kₘ value calculated for any other UGT enzyme. Preferably, the Kₘ of the tested compound calculated for UGT2B10 is at least 2-fold less (i.e. at least 2-fold selectivity), more preferably at least 5-fold less (i.e. at least 5-fold selectivity), for example at least 10-fold less (i.e. at least 10-fold selectivity) compared to the calculated Kₘ value for any other UGT enzyme. It is also preferred that the Kₘ value for UGT2B10 of the compound identified as a UGT2B10 substrate is close to the Kₘ value for human liver microsomes indicating that the UGT2B10-catalyzed reaction is relevant in human liver. For example, the calculated Kₘ value for UGT2B10 is within the range of 60-140%, preferably within the range of 70-130%, more preferably within the range of 80-120%, of the calculated Kₘ value for human liver microsomes. Example 2 of the present application describes the procedure of estimating the selectivity of nicotine glucuronidation for UGT2B10 over UGT1A4.

For the purpose of identifying a compound which acts as UGT2B10 modulator, recombinant cells that express UGT2B10 are prepared as described above. The cells expressing UGT2B10 are suspended in water and homogenized using known procedures. The incubation mixture is prepared by mixing recombinant UGT2B10 homogenate and uridine-5-diphosphoglucuronic acid (UDP-GA) as a glucuronic acid source in a suitable buffer such as phosphate buffer pH 7.4. Various concentrations of the tested modulator are added to the mixture along with a compound that is known to be metabolized by UGT2B10, i.e. the substrate, and the mixture is incubated at 37° C. for a period needed for the glucuronidation reaction to occur, typically from 0.5 to 2 hours. The reaction is terminated as described above. Following centrifugation, the supernatants are analyzed and quantified for the presence of glucuronidated derivative of the substrate as described above. The UGT2B10 enzyme activity towards the substrate, in the absence and presence of the modulator, is calculated as the amount of glucuronide formed (pmol) divided by incubation time (min) and protein amount (mg/incubation). The effect of the modulator, inhibition or activation, on the enzyme activity towards the substrate is calculated as % enzyme activity at each modulator concentration compared to the enzyme activity measured in the absence of the modulator. IC₅₀, inhibitor concentration that inhibits the activity by 50%, is used as the measure of the inhibitory effect, and the activation effect is calculated as % increase in enzyme activity. Example 3 of the present application describes the procedure of identifying an UGT2B10 modulator.

The efficacy of the UGT2B10 modulator in modifying the pharmacokinetics of a pharmacologically active agent that undergoes direct N-glucuronidation by UGT2B10 can be studied in a human subject by administering the pharmacologically active agent to a human subject alone or in combination with the UGT2B10 modulator (acting as an enzyme inhibitor or activator). The route of administration can be e.g. intravenous or oral for both agents. Venous blood samples are collected prior to dosing and at pre-determined time points (e.g. at 0.5, 1, 2, 4, 6, 8, 12 and 24 hours) post-dose. Plasma is separated from whole blood using standard centrifugation techniques and the samples are analysed for the pharmacologically active agent. Appropriate pharmacokinetic parameters (Cₘₐₓ, Cₘᵢₙ, AUC, T_{1/2}) are calculated using non-compartmental analysis. The data for the administration of the pharmacologically active agent alone or in combination with the UGT2B10 modulator are compared using standard statistical methods. The UGT2B10 modulator, characterised as an enzyme inhibitor, is considered to be effective if it results in improved pharmacokinetics, e.g. in an increased AUC of the pharmacologically active agent or a reduction in the plasma level variability of the pharmacologically active agent. The UGT2B10 modulator, characterised as an enzyme activator, is considered to be effective if it results in e.g. a decrease in the AUC of the pharmacologically active agent.

According to one embodiment of the present invention, the pharmacologically active agent that is useful for the treatment or prevention of a disease or a condition and the UGT2B10 modulator is administered to a human subject for providing improved pharmacokinetics of said agent. The active agent and the UGT2B10 modulator can be provided, according to one embodiment of the invention, in the form of a pharmaceutical combination for simultaneous, separate or sequential administration.

The UGT2B10 modulator is administered to a human subject in an amount sufficient to provide improved pharmacokinetics of the active agent (as measured by AUCs or otherwise as described herein) compared to the pharmacokinetics obtained in the absence of the UGT2B10 modulator. For example, when active agent is useful for the treatment or prevention of a disease or a condition, the UGT2B10 modulator can be administered in an amount that provides Cₘₐₓ, Cₘᵢₙ, AUC and/or T_{1/2} values for the active agent that are at least 1.1 fold, at least 1.2 fold, at least 1.3 fold or at least 1.5 fold, compared to values obtained in the absence of the UGT2B10 modulator. The actual amount of the UGT2B10 modulator to be included in the particular combination or composition will vary depending on the modulator's activity and the pharmacologically active agent used. The amount of the UGT2B10 modulator can be optimized by measuring the AUC of the active agent after administering the active agent with various doses of the modulator. The ratio of the UGT2B10 modulator to the pharmacologically active agent in a particular combination or composition is in the range of 0.01-100:1, typically in the range of 0.1-10:1, and more typically in the range of 0.5-2:1.

According to one embodiment of the invention, the pharmacologically active agent is co-formulated together with the UGT2B10 modulator to a pharmaceutical composition. The pharmaceutical composition comprising the pharmacologically active agent and the UGT2B10 modulator can be formulated into pharmaceutical dosage forms using the principles known in the art. They are given to a subject as such or preferably in combination with suitable pharmaceutical excipients in the form of tablets, granules, capsules, suppositories, emulsions, suspensions or solutions. Choosing suitable excipients for the composition is routine for those of ordinary skill in the art. It is evident that suitable carriers, solvents, gel forming ingredients, dispersion forming ingredients, antioxidants, colours, sweeteners, wetting compounds, release controlling components and other ingredients normally used in this field of technology may be also used.

The active agent and the UGT2B10 modulator may be formulated in the same pharmaceutical formulation. Alternatively, the active ingredients are formulated as separate pharmaceutical dosage forms. The combination of the pharmaceutical dosage forms may be packaged as a single pharmaceutical combination product or kit, optionally together with a package insert instructing to the correct use of the combination product.

The following examples are illustrative only and are not intended to limit the scope of the present invention.

### EXPERIMENTS

### Example 1

### Identifying Substrates for UGT2B10 In Vitro

Recombinant human UGT2B10 were produced in baculovirus-infected insect cells and used as cell homogenate after suspending the cells with water. The glucuronidation rates were determined by incubating the substrates, nicotine and cotinine, with the enzyme UGT2B10 using uridine-5′-diphosphoglucuronic acid (UDP-GA) as a cofactor. The reaction mixtures were incubated at +37° C. for 1-2 hours. The samples were analyzed by high-performance liquid chromatography-mass spectrometry (HPLC-MS). The formed glucuronides were quantified using authentic glucuronide standards. The enzyme activity of UGT2B10 towards the tested compound was calculated as pmol glucuronide formed divided by incubation time (min) and protein amount (mg/incubation).

The results for two model substrates, nicotine and cotinine, are shown in Table 1. Both compounds were substrates for UGT2B10.

TABLE 1
UGT2B10 enzyme activities for nicotine and cotinine in
vitro at 2 mM substrate concentration.

| | UGT2B10 activity |
|---|---|
| Compound | (pmol/min/mg prot.) |
| Nicotine | 2.9 |
| Cotinine | 12.7 |

### Example 2

### The Selectivity of Nicotine Glucuronidation for UGT2B10 Over UGT1A4 In Vitro

Recombinant human UGTs were produced in baculovirus-infected insect cells. UGT1A4 was used after the isolation of the microsomal membranes, which is a common purification step for UGT enzymes. However, UGT2B10 was used as cells homogenate after the observation that the activity of this enzyme sharply declined upon the isolation of the microsomal membranes. Human liver microsomes (HLM) were obtained from BD Biosciences (Woburn, Mass., USA).

The UGT enzyme and HLM activities were determined by incubating various concentrations (0.002-2 mM) of nicotine with recombinant human UGT2B10 and UGT1A4, and HLM using uridine-5′-diphosphoglucuronic acid (UDP-GA, Sigma) as a cofactor. The reaction mixtures were incubated at +37° C. for 1-2 hours. The samples were analyzed by high-performance liquid chromatography-mass spectrometry (HPLC-MS). Nicotine glucuronide was quantified using authentic standard and nicotine glucuronide (methyl-D3) (Toronto Research Chemicals, Canada) as an internal standard. The enzyme activities for UGTs and human liver micorosomes at various concentrations of the tested compound were calculated as the amount of glucuronide formed (pmol) divided by incubation time (min) and protein amount (mg/incubation). Enzyme kinetic parameter Kₘ was estimated fitting the enzyme activity data to Michaelis-Menten equation using SigmaPlot Enzyme Kinetics Module v. 1.1 (SPSS Inc., Chicago, Ill.) software.

The results are shown in Table 2. The Kₘ of nicotine glucuronidation for UGT2B10 is 8-fold less compared to UGT1A4 indicating clear selectivity for UGT2B10 over UGT1A4. In addition, the measured Kₘ for UGT2B10 is very close to the Kₘ measured for human liver microsomes showing the importance of UGT2B10 in human hepatic glucuronidation of nicotine.

TABLE 2
Kₘ values of nicotine glucuronidation for UGT2B10, UGT1A4 and
human liver microsomes (HLM).

| | Kₘ, UGT2B10 | Kₘ, UGT1A4 | Kₘ, HLM |
|---|---|---|---|
| Compound | (mM) | (mM) | (mM) |
| Nicotine | 0.29 | 2.4 | 0.33 |

### Example 3

### Identifying UGT2B10 modulators in vitro

Recombinant human UGT2B10 was produced in baculovirus-infected insect cells and used as cell homogenate after suspending the cells with water. The UGT enzyme activities were determined by incubating the known UGT2B10 substrate, nicotine (0.2 mM), in the absence and presence of (S)-4-[1-(2,3-dimethylphenyl)-ethyl]-3H-imidazole (levomedetomidine) (0.001, 0.01, 0.1 and 1 mM) with UGT2B10 using uridine-5′-diphosphoglucuronic acid (UDPGA) as a cofactor. The reaction mixtures were incubated at +37° C. for 2 hours. The samples were analyzed by high-performance liquid chromatography-mass spectrometry (HPLC-MS). The formed glucuronide was quantified using authentic glucuronide standard. The UGT2B10 enzyme activity towards the tested compound was calculated as pmol glucuronide formed divided by incubation time (min) and protein amount (mg/incubation). Levomedetomidine was identified as UGT2B10 inhibitor for nicotine glucuronidation. The inhibitory effect of the levomedetomidine on the UGT2B10 enzyme activity for nicotine was calculated as % remaining enzyme activity at each inhibitor concentration compared to the measured enzyme activity in the absence of the inhibitor. IC₅₀, the half maximal inhibitory concentration, was used as the measure of the inhibitory effect.

Levomedetomidine inhibited the UGT2B10 enzyme activity for nicotine, the observed IC₅₀ value was 0.013 mM. In comparison, the IC₅₀ value for human liver microsomes was 0.026 mM, and the IC₅₀ value for UGT1A4 was >1 mM indicating that the inhibition was selective for UGT2B10 over UGT1A4, and the inhibition is relevant in human liver.

1. A method for modifying the pharmacokinetics of a pharmacologically active agent that undergoes direct N-glucuronidation by UDP-glucuronosyltransferase UGT2B10 in a human subject, comprising administering an effective amount of a UGT2B10 modulator to said human subject.

2. A pharmaceutical combination comprising a pharmacologically active agent that is useful for the treatment or prevention of a disease or a condition and that undergoes direct N-glucuronidation by UDP-glucuronosyltransferase UGT2B10 and a UGT2B10 modulator in an amount that provides improved pharmacokinetics of said pharmacologically active agent.

3. A method for identifying a compound which is directly metabolized by UDP-glucuronosyltransferase UGT2B10, comprising
(a) providing a homogenate of recombinant cells that express UGT2B10,
(b) incubating the compound to be tested with said homogenate together with a glucuronic acid source,
and
(c) determining whether N-glucuronidated derivative of the compound was formed.

4. A method for identifying a compound which acts as UGT2B10 modulator, comprising
(a) providing a homogenate of recombinant cells that express UGT2B10,
(b) incubating a UGT2B10 substrate with said homogenate together with a glucuronic acid source in the presence and in the absence of the compound to be tested,
(c) quantifying the amount of N-glucuronidated derivative of said UGT2B10 substrate that was formed in the presence and in the absence of the tested compound,
and
(d) determining whether the N-glucuronidation of said UGT2B10 substrate was inhibited or activated by the tested compound.
